(19) United States
(12) Patent Application Publication
(10) Pub. No.: US 2020/0407331 A1
(43) Pub. Date: Dec. 31, 2020

(54) UNIVERSAL BUILDING BLOCKS FOR RADIOLABELING

(71) Applicant: HELMHOLTZ-ZENTRUM DRESDEN - ROSSENDORF E.V., Dresden (DE)

(72) Inventors: Hans-Jürgen PIETZSCH, Heidenau (DE); Martin WALTHER, Ottendorf-Okrilla (DE)

(73) Assignee: HELMHOLTZ-ZENTRUM DRESDEN - ROSSENDORF E.V., Dresden (DE)

(21) Appl. No.: 16/975,624

(22) PCT Filed: Feb. 28, 2019
(86) PCT No.: PCT/EP2019/054954

§ 371 Date: Aug. 25, 2020

(30) Foreign Application Priority Data
Feb. 28, 2018 (EP) 18159078.7

(51) Int. Cl. C07D 249/04; C07C 229/14; C07B 59/00

(52) U.S. Cl. CPC C07D 249/04; C07C 2601/14; C07B 59/004; C07C 229/14

(57) ABSTRACT

The present invention describes novel chelators (multidentate ligands) and precompounds for complexation of radiometals and non-radioactive counterparts, for use in radiopharmacy. The invention includes a process and a kit involving such chelators.

Active moieties directing to a pharmaceutical target (such as peptides or proteins) can be attached to the chelator very easily via the so called “click-chemistry” forming a triazole-ring moiety. The aromatic triazole-nitrogen itself acts as a new and “soft” nucleophilic site enabling for complexation of various radiometals or non-radioactive counterparts. The chelators are capable of fast complexation at low temperature.

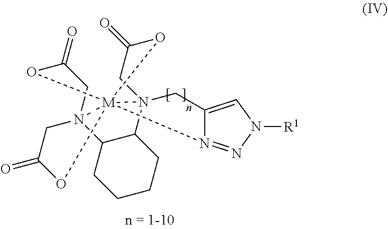

The present invention generally relates to the field of radiopharmacy. In particular, it is useful for radiolabeling of temperature-sensitive biomolecules (such as peptides or proteins) for therapeutic or diagnostic use.

### PRIOR ART

Public knowledge deals with the well-known chelators NOTA (originally mainly for ⁶⁸Ga) and NODA both requiring high temperature for complexation of Al¹⁸F (>100° C.):

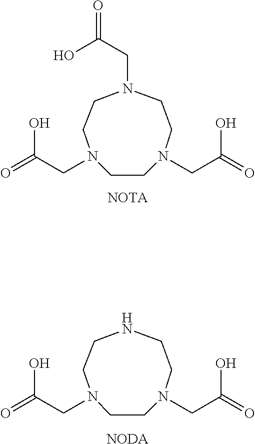

NOTA and NODA are currently used as chelators for ⁶⁸Ga and Al¹⁸F.

WO2016/065435A2 and Cleeren et al. (2016) both describe a one-step complexation procedure for complexation of {Al¹⁸F}²⁺ in aqueous medium using alternative chelators enabling complex formation at much lower temperatures (<40° C.). Stability and biodistribution was investigated. Novel chelators were developed and tested. It was found that, for chelation of {Al¹⁸F}²⁺, open variants of that chelator ligands were much faster and enabled for chelation at lower temperatures compared to macrocyclic ligands as NOTA and NODA. The term “open” refers to non-cyclic amino groups (which later act as electron donating sites for complexation of the radionuclide metals). Modification through functionalization of those chelators used in that prior art document needs reactive groups to attach pharmaceutically active molecules, proteins, biological binding moieties or particles.

For complexation of different radiometals or the corresponding non-radioactive counterparts, each one requires chemical modification and optimization of the chelator's structure, depending on the preferred valency of the radiometal or the corresponding counterpart.

The attachment of pharmaceutically active molecules, proteins, biological binding moieties or particles to the chelator needs time-consuming, laborious modification of the chelator molecules often via synthesis of activated esters etc.

Vanasschen et al. (2017) deals with a symmetric cyclohexyl-based chelator with two exocyclic nitrogen and four “hard” carboxylate moieties for complexation of the Mn^{II} ions.

### Technical Problem

The problem to be solved is to provide novel chelators (multidentate ligands). In particular, the chelators should be feasible for various radiometals including the non-radioactive counterparts. Further, it should be possible to attach further target seeking molecules like peptides, proteins and particles easily. Further the chelators should be suitable for fast complexation ideally at low temperature (<40° C.).

### Solution

The technical problem is solved through the invention by applying a substituted triazole group (formulas II, III and IV) that provides both an additional aromatic nitrogen for weak coordination and the possibility for easy functionalization. First, the chelators become applicable for fast complexation of various radiometals (or the non-radioactive counterparts) without the need of further modification of the chelator. Second, the functionalization can be achieved via the triazole moiety very easily.

A chelator, in complexation chemistry, is a multidentate ligand that bears more than one free electron pair that can act as an electron donating site for complexation of a central atom. In this case the ligand is called chelator. In contrast, a (monodentate) ligand is a molecule with one free electron pair acting in complexation. According to the invention a ligand is comprising carbonyl (—CO), isocyanide (R′NC—), ¹⁸F and oxygen (═O), with R′ comprising aliphatic substituents with 1 to 10 C-atoms or aromatic substituents with 5 to 10 C-atoms. Preferably, for Tc (technetium) such a ligand is CO or ═O and for Al (aluminium), Ca (calcium) or Mg (magnesium) such a ligand can be F or radionuclides thereof, i.e. Al¹⁸F, ^{99m}Tc(O), ¹⁸⁶Re(O), ¹⁸⁸Re(O).

Scheme 1 illustrates the synthetic pathway from an alkyne precompound (I) which forms the new triazole ring in formula (II) together with a substituted azide (R¹—N₃) via copper catalyzed azide-alkyne cycloaddition, wherein R and R¹ are defined later in the invention. Deprotection of the carboxyl groups yields chelator (III). Chelator (III) is now capable of complexing various radionuclides (see formula IV).

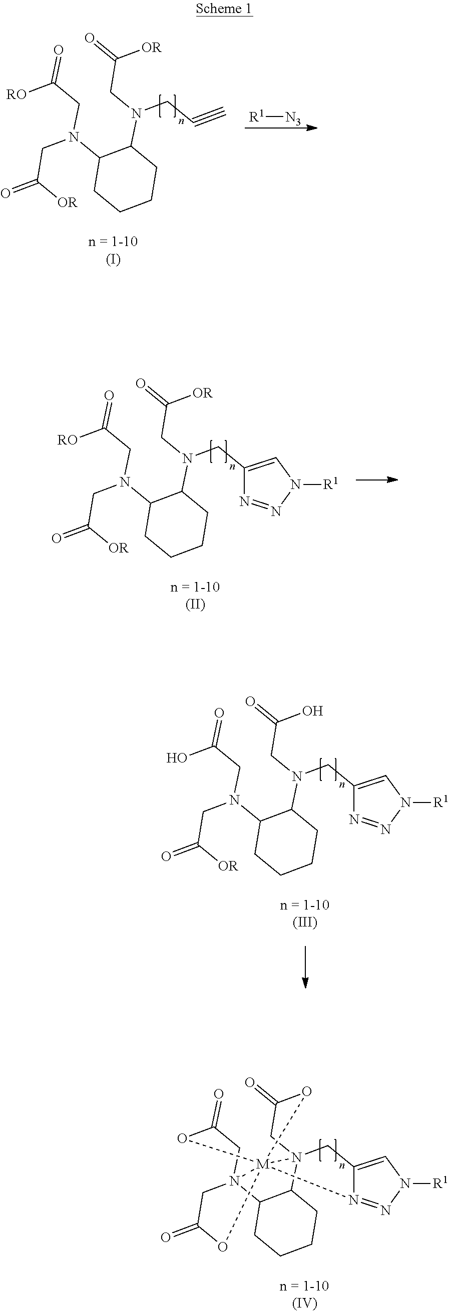

The nitrogen of the introduced triazole moiety, particularly, is promoting complexation due to its stabilization by a mesomeric effect (+M effect) and, hence, is generally known as a soft nucleophilic moiety. Remaining free carboxylate groups are known as hard nucleophiles. By adding a further soft nucleophilic side the applicability in complexation of different radionuclides is enhanced. Hence, the chelator is applicable for various radiometals and/or non-radioactive counterparts and there is no need to further modify the chelator depending on the radiometal/non-radioactive counterpart (for later complexation).

Furthermore, the problem is solved, as this triazole group can be introduced by the so called “click chemistry” which is a well known versatile, broadly applicable and very easy synthetic method of organic chemistry. Any target-directing functional groups (active moiety) can be easily attached to the chelator by means of that click-chemistry. In the present invention those target-directing groups were attached to the triazole moiety by a linker (defined below).

Click-chemistry is defined in this case as a copper-mediated reaction of an alkyne with an organic azide to form a five-membered triazole ring (also well known as Copper catalyzed Azide-Alkyne Cycloaddition CuAAC).

The active moiety is a molecule. Such active moiety is directed to a biological target. It is the part of a drug that makes the drug work the way it does. The molecule or ion, which is responsible for the physiological or pharmacological action of the drug substance, excluding those appended portions of the molecule that cause the drug to be an ester, salt (including a salt with hydrogen or coordination bonds), or other noncovalent derivative (such as a complex, chelate, or clathrate) of the molecule.

The active moiety is in particular selected from peptides, oligonucleotides, proteins, enzymes antibodies, antibody fragments, macromolecules, nanoparticles and small organic molecules.

According to the invention suitable linkers are short groups with 1 to 20 C-Atoms, that can be substituted (e. g. with polar groups, like OH or ═O) or unsubstituted. In particular, some (1-5) C-Atoms can be substituted by heteroatoms, preferably N or O.

Preferably the linkers are short groups with 5 to 10 C-Atoms, that can be substituted or unsubstituted and that can be substituted by heteroatoms as described above.

One object of the invention is a compound of formula (I) and salts thereof as a starting compound for building up the triazole moiety of the present invention via so called “click-chemistry”:

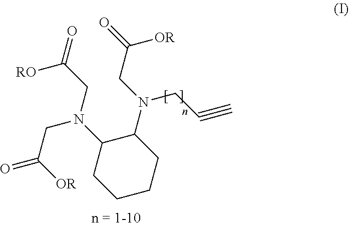

wherein each R is a carboxyl protecting group. Such carboxyl protecting groups are well known to the person skilled in the art, e.g. tert-butyl, ethyl or methyl.

According to the invention this compound of formula (I) or salts thereof are used for preparation of a complex for use in radiopharmacy.

Preferably, this compound of formula (I) or salts thereof is used for preparation of a complex of formula (IV).

Another object of the invention is the use of a compound of formula (I) or salts thereof for preparation of compounds of formula (II) or formula (III).

Another object of the invention is the final compound (II) and salts thereof

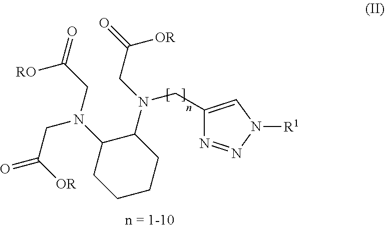

wherein R¹ is -alky, -aryl or -heteroaryl, or a linker attached to either an active moiety directly or a group for functionalization,

wherein each R is a carboxyl protecting group. Such carboxyl protecting groups are well known to the person skilled in the art, e.g. tert-butyl, ethyl or methyl.

The term alkyl comprises unsubstituted and substituted alkyl residues with in a particular 1 to 15, preferably 1 to 10, C-atoms. The term substituted alkyl comprises in particular alkyl with aliphatic or aromatic side chains with up to 7 C-atoms or alkyl substituted with halogens or polar groups, like OH, SH, COOH.

The term aryl comprises unsubstituted and substituted aryl residues with preferably 5 to 20 C-atoms, like phenyl. The term substituted aryl comprises in particular aryl with aliphatic side chains with up to 5 C-Atoms or aryl substituted with halogens or polar groups, like OH, SH, COOH.

The term heteroaryl is defined as above for aryl, with the sole difference that 1 to 4 C-Atoms in the aryl are exchanged by heteroatoms chosen from N, S and O, preferably N.

Suitable groups for functionalization are known in the art. The term comprises groups that allow the binding of active moieties by a chemical reaction or by a strong physical binding. Groups for functionalization in particular comprise amino or sulfhydryl or carboxyl reactive groups, but also thiols or thiolates e. g. for binding of gold particles. Suitable groups are in particular selected from reactive esters like succinimidyl ester (NHS), azides (—N₃), isothiocyanates (—NCS), isocyanates (—NCO) and maleimides.

Another object according to the invention is the use of compound (II) or salts thereof to form a complex comprising compound (III) as chelator, and a metal or salts thereof.

Preferably those metal is selected from Al, Ca or Mg or radionuclides ¹¹¹In, in ⁶⁷Ga, ⁶⁸Ga, ⁶⁰Cu, ⁶¹Cu, ⁶²Cu, ⁶⁴Cu, ⁶⁷Cu, ^{99m}Tc, ¹⁸⁶Re, and ¹⁸⁸Re and the complex is used for radiopharmacy.

Most preferably those metal is selected from Al, Ca or Mg or radionuclides ⁶⁷Ga, ⁶⁸Ga, ⁶⁰Cu, ⁶¹Cu, ⁶²Cu, ⁶⁴Cu, ⁶⁷Cu, ^{99m}Tc, ¹⁸⁶Re, and ¹⁸⁸Re and the complex is used for radiopharmacy.

In a more preferred embodiment this complex further comprises CO, R′NC, ¹⁸F and ═O as ligands, especially CO, R′NC and ¹⁸F.

Radiopharmacy is defined as a scientific area that involves preparation of radioactive materials for patient administration that will be used to diagnose and treat specific diseases in nuclear medicine. It generally involves the practice of combining a radionuclide tracer with a pharmaceutical component that determines the biological localization in the patient.

Another object of the invention is a complex and salts thereof according to formula (IV)

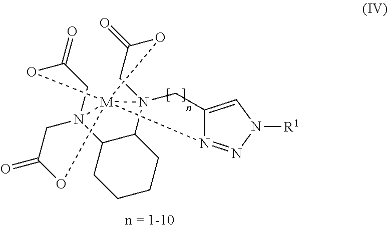

wherein M is comprising a metal, a radiometal and a radionuclide and optionally further comprising CO, R′NC, ¹⁸F and ═O ligands, or preferably further CO— or R′NC— or ¹⁸F-ligands.

wherein R¹ is defined as above,

wherein R′ is alkyl or aryl, preferably with 1 to 7 C-atoms,

wherein two or more of those interactions (shown as dashed lines in formula (IV)) between M and the electron donating atoms are present depending on the radiometal (or non-radioactive counterpart) and its preferred valency,

This variability in the number of interactions and the degree of softness/hardness of the electron donating atoms is one key of the solution of this invention to enable complexation of radiometals (or corresponding non-radioactive counterparts) of various valency states.

This complexes of formula (IV) can equally be described as a complex comprising a metal M, and a chelator, wherein the chelator is a compound of formula (III)

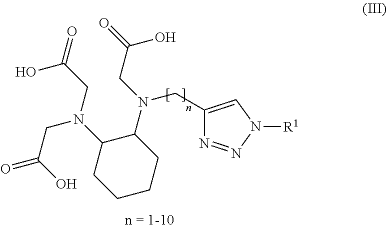

or salts thereof,

with the definitions equal to those above (for complex of formula (IV)).

In another embodiment of the invention, M according to the complex of formula (IV) is selected from Al, Ca, Mg, ¹¹¹In, ⁶⁷Ga, ⁶⁸Ga, ⁶⁰Cu, ⁶¹Cu, ⁶²Cu, ⁶⁴Cu, ⁶⁷Cu, ^{99m}Tc, ¹⁸⁶Re, and ¹⁸⁸Re, preferably from Al, ⁶⁷Ga, ⁶⁸Ga, ⁶⁰Cu, ⁶¹Cu, ⁶²Cu, ⁶⁴Cu, ⁶⁷Cu, ^{99m}Tc, ¹⁸⁶Re, and ¹⁸⁸Re.

This complex may further comprise CO, R′NC, ¹⁸F (like in Al—F¹⁸, Mg—F¹⁸ and Ca—F¹⁸) and ═O as ligands, preferably CO, R′NC and ¹⁸F, wherein R′ is defined above.

In a preferred embodiment the latter complex is used in the diagnosis or therapy of cancer.

The invention also comprises corresponding diastereomers and enantiomers of compounds of the invention, and mixtures thereof.

Object of the invention are also salts and solvates of the compounds of the invention.

Preferred are pharmaceutically acceptable salts comprising salts of carboxylic acids, mineral acids, hydroxycarbonic acids, sulfonic acids, boronic acids and salts of common bases, e.g. alkali metal salts, alkaline earth metal salts, ammonia salts. Salts, which are not pharmaceutically acceptable, but which are suitable for isolation or purification of compounds of the invention are also comprised. Solvates are complexes of compounds of the invention with water or other solvent molecules or mixtures thereof.

Another object of the invention is the use of chelator of formula (II) or (III) or the use of complex of formula (IV) for preparation of a medicament, in particular for the treatment of cancer.

The invention also comprises the medical use of chelator compound (III) or complex of formula (IV) for diagnostics of, e.g. the heart, by administration of the radioactive metal complex of formula (IV) to a patient. Intravenous administration of the radioactive metal complex preferably leads to a rapid mycocardial uptake of the radioactive metal complex, and rapid blood, liver and lung clearance, so that the radioactive metal complex can be used for radiopharmaceutical diagnostic imaging, preferably suitable for in vivo heart imaging.

Another object of the invention is also a non-radioactive kit comprising compound (III) or salts thereof for use in radiopharmacy.

In a preferred embodiment this non-radioactive kit is used for the for the preparation of a radiopharmaceutical composition, comprising at least one container, wherein one container contains:
(i) a stabilized form of compound (III) or salts thereof according to the invention.

Preferably the kit comprises one or several additional ingredients selected from:
(ii) preservative,
(iii) agents for pH adjustment, and
(iv) fillers

which are defined below.

In the present invention the kit preferably comprises of one sterile container. The sterile containers enable maintain sterility of the pharmaceutical formulations, facilitate transportation and storage, and allow administration (e. g. intravenous) of the pharmaceutical formulations without prior sterilization step. Preferably, the container is a sealed and sterilized container selected from a group comprises vial, syringe bottle, or ampoule, wherein the container may come in of various sizes and capacities.

The non-radioactive kit according to the invention can be formulated as single dose kit or multi dose kit. Preferably, the kit is a multi dose kit, which comprises sufficient material for multiple patient doses from the same radiopharmaceutical composition.

The labelling reaction to form a complex of compound (III) can be performed under acidic conditions, preferably above pH 4. An acid solution is generally acceptable for human or mammalian administration. The non-radioactive kit according to the invention preferably comprises an agent for pH adjustment to adjust the pH of a radiopharmaceutical composition within preferred ranges (approximately pH 4.0 to 10.0), more preferably nearer to or within the physiological pH range, which is preferred for human or mammalian administration and lies between pH 6 and 9.5, preferably between 7.5 and 9.0.

Preferably, agents for pH adjustment or pH regulating agents comprise sterile solutions or sterile powders of the salts. The agent for pH adjustment preferably comprises a member selected from the group consisting of pharmaceutically acceptable buffers or agents for pH adjustment, such as citrate, hydrogen and/or sodium carbonates, hydrogen phosphates, TRIS, tricine or mixtures thereof.

A preferred agent for pH adjustment for the kits according to the invention is a salt of carbonic acid, like carbonate or hydrogen carbonate, more preferably sodium hydrogen carbonate (NaHCO₃). In case the non-radioactive kit according to the invention comprises a buffer or agent for pH adjustment, the buffer or agent for pH adjustment is preferably not in the same container as the stabilized form of compound (II) according to the invention.

E.g. for technetium labeling first the stabilized form of compound (II) according to the invention mixed with additional ingredients is dissolved by adding a solution of the buffer or an agent for pH adjustment. Subsequently, the pertechnetate solution to form the technetium-compound(II) complex is added. Likewise, the optional buffer or agent for pH adjustment can be added to the radiopharmaceutical composition after the radiolabeling procedure is finished.

The term “filler” as used herein refers to a pharmaceutically acceptable bulking agent, which may facilitate material handling during production of a kit or a radiopharmaceutical composition thereof. Suitable fillers include inorganic salts such as sodium chloride, and water soluble sugars or sugar alcohols such as sucrose, maltose, D(−)-mannitol or trehalose. Certain buffer salts or agents for pH adjustment may also function as bulking agents. The mass fraction of the filler used in the non-radioactive kits of the present invention is chosen freely, but is typically in the range between 1 to 30 mg, more preferably between 5 to 25 mg based on the total weight of the stabilized form of compound (II).

In addition, if desired or necessary, the non-radioactive kit of the present invention optionally further contains pharmaceutically acceptable preservatives to prevent decomposition of organic matter, e. g. due to microbial contamination, so that the kit can be stored for long periods of time. The preservatives are preferably selected from the group consisting of ascorbic acid, benzyl alcohol, cresol; cetrimide, thiomersal, phenol and the parabens (e. g. methyl, ethyl, propyl or butyl paraben or mixtures thereof).

In a preferred embodiment of the invention the index n in all chemical structures shown is 1-5, especially 1-3, mostly preferred it is 1.

For the embodiment with n=1, the formulas (I), (II), (III) and (IV) are exactly as the following formulas (Ia), (IIa), (IIIa) and (IVa):

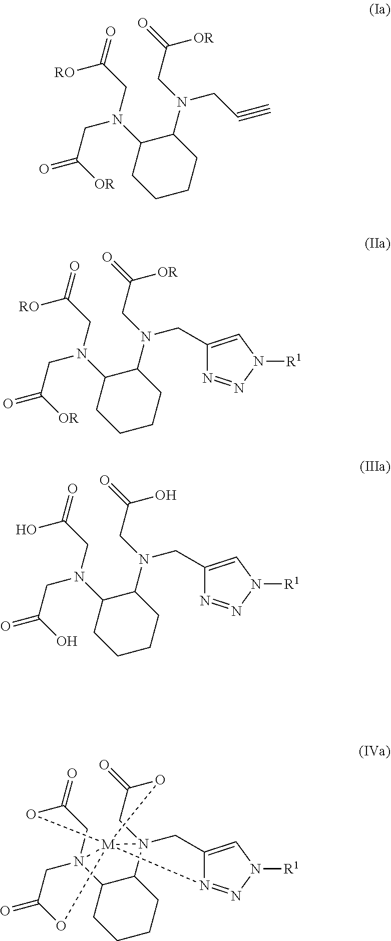

Preferably, the compounds according to formula (III), and (IIIa) correspondingly, are selected from

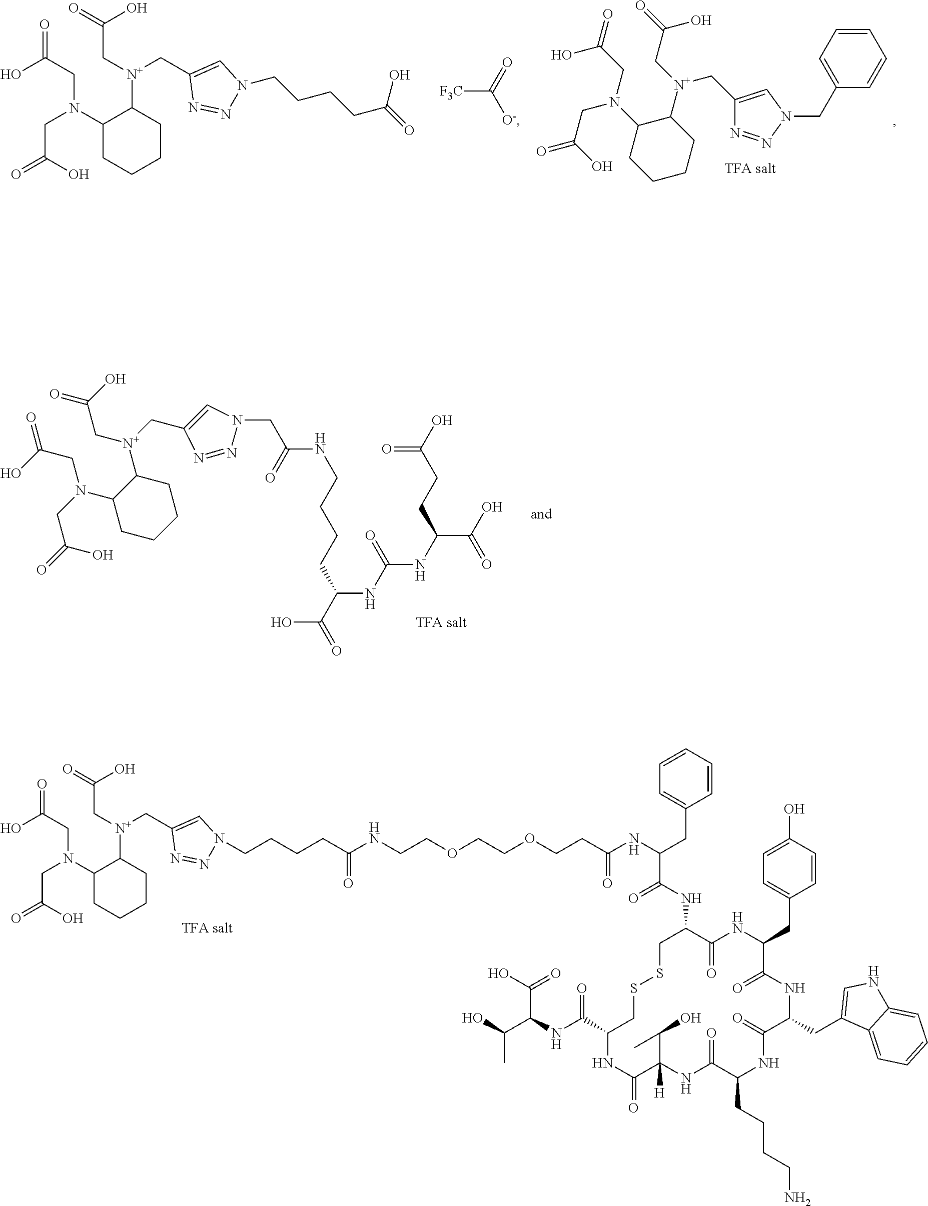

Preferably, the compound according to formula (II), and (IIa) correspondingly, is

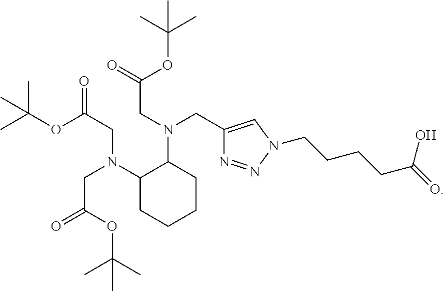 [SMILES: CC(C)(C)OC(=O)CN(CC(=O)OC(C)(C)C)C1CCCCC1N(CC(=O)OC(C)(C)C)Cc1cn(CCCCC(=O)O)nn1]

Preferably, the compound according to formula (I), and (Ia) correspondingly, is

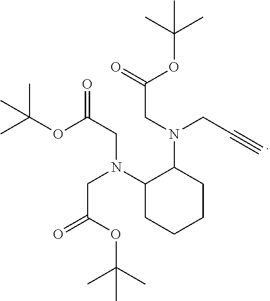 [SMILES: C#CCN(CC(=O)OC(C)(C)C)C1CCCCC1N(CC(=O)OC(C)(C)C)CC(=O)OC(C)(C)C]

When it is stated during the text “in one embodiment” or “another embodiment” or similar expression, this does not exclude the possibility that the embodiments can be combined.

The invention is illustrated by the following embodiments without being limited to these:

General Method for Preparation of Chelators (Compound (III) from Compound (I))

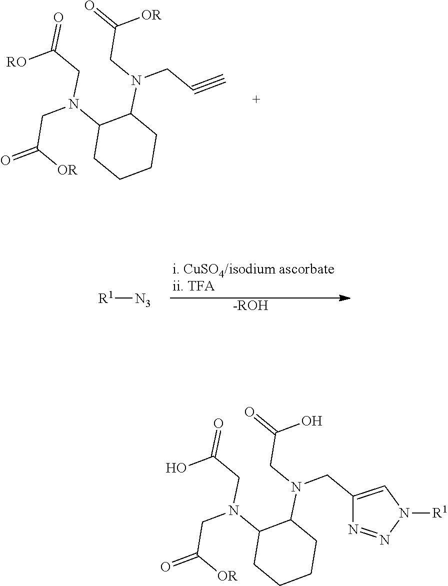

To a solution of alkyne (40 μmol) and substituted azide (80 μmol) in tert-butyl alcohol (750 μl) TBTA (2.5 μmol) was added. After brief stirring, 1 M sodium ascorbate (50 μl) and 0.1 M CuSO₄ (100 μl) solutions were added and the mixing was continued at room temperature for 24 h. The solution was then diluted with ethyl acetate (10 ml) and extracted with aqueous 0.05 M EDTA (3×5 ml) and then 1 M NH₄OH (3×5 ml) solutions. The organic phase was dried (Na₂SO₄), filtered and evaporated under reduced pressure to afford the crude product, which was used without further purification. The crude product, was dissolved in a mixture of TFA (450 μl) and H₂O (50 μl) and stirred for 5 h at rt. Then the reaction mixture was added dropwise to ice cooled Et₂O (10 ml). The supernatant was decanted and the precipitate was washed twice with cold Et₂O.

### General Methods for Preparation of Compound (I), Compound (II) and Compound (III)

### Preparation of tert-Butyl 2-[[2-[bis(2-tert-butoxy-2-oxo-ethyl)amino]cyclohexyl]-prop-2-ynyl-amino]acetate (1)

The synthesis of the mono alkyne substituted cyclohexane compound 1 as starting material is carried out by a standard alkylation method using tert-butyl 2-[[2-[bis(2-tert-butoxy-2-oxo-ethyl)amino]cyclohexyl]amino]acetate (Mohamadi et al. (2017) and Gale et al. (2015)) as amine and propargyl bromide as alkylation agent. Additionally, were used trimethylamine (TEA) as a base and acetonitrile as solvent.

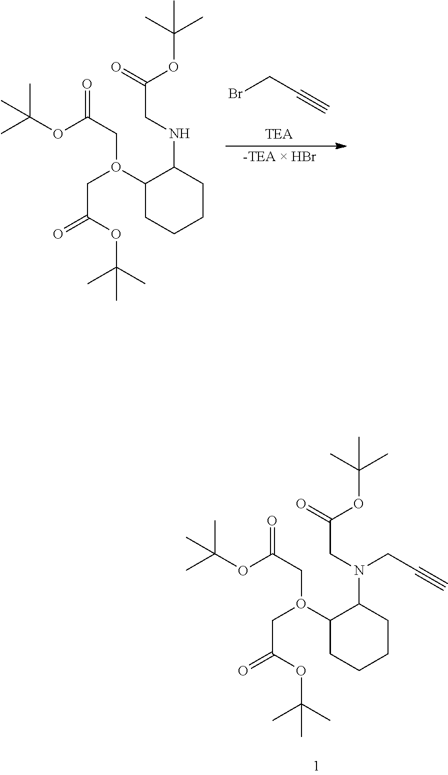

¹H NMR (400 MHz, CDCl₃) δ 3.69, 3.50, 3.39, 2.69, 2.17, 2.03, 1.67, 1.46, 1.45, 1.25, 1.13. ¹³C NMR (101 MHz, CDCl₃) δ 171.74, 171.21, 81.87, 80.55, 80.28, 77.20, 72.21, 62.88, 62.82, 53.71, 52.32, 40.01, 29.92, 28.27, 28.14, 28.12, 25.65, 25.59.

Chemical Formula: C₂₇H₄₆N₂O₆, Molecular Weight: 494.66 g/mol, ESI⁺ m/z: 495 [M+H]⁺.

### Preparation of tert-Butyl 2-[[2-[bis(2-tert-butoxy-2-oxo-ethyl)amino]cyclohexyl]-hex-2-ynyl-amino]acetate (1′)

The synthesis of the mono alkyne substituted cyclohexane compound 1′ as starting material is carried out by a standard alkylation method using tert-butyl 2-[[2-[bis(2-tert-butoxy-2-oxo-ethyl)amino]cyclohexyl]amino]acetate (Mohamadi et al. (2017) and Gale et al. (2015)) as amine and 6-chloro-1-hexyn as alkylation agent. Additionally, were used trimethylamine (TEA) as a base and acetonitrile as solvent.

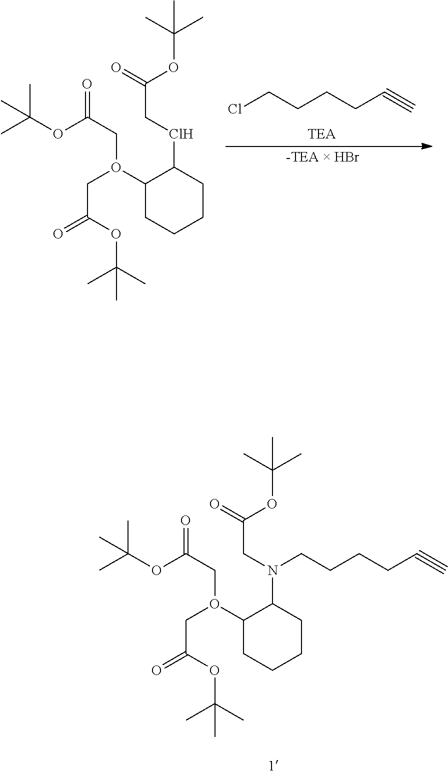

### Preparation of 5-[4-[[[2-[bis(2-tert-butoxy-2-oxo-ethyl)amino]cyclohexyl]-(2-tert-butoxy-2-oxo-ethyl)amino]methyl]triazol-1-yl]pentanoic acid (2)

To a solution of 20 mg alkyne 1 (40 μmol) and 11 mg 5-azidopentanoic acid (80 μmol) in tert-butyl alcohol (750 μl) 1.3 mg TBTA (2.5 μmol) was added. After brief stirring, 1 M sodium ascorbate (50 μl) and 0.1 M CuSO₄ (100 μl) solutions were added and the gentle mixing was continued at room temperature for 24 h. The reaction mixture was then diluted with ethyl acetate (10 ml) and extracted three times with aqueous 0.05 M EDTA (3×5 ml) and then 1 M NH₄OH (3×5 ml) solutions to remove copper salts. The organic phase was dried (Na₂SO₄), filtered and evaporated under reduced pressure to afford the crude product, which was used without further purification. Yield: 20.1 mg (31 μmol; 77%)

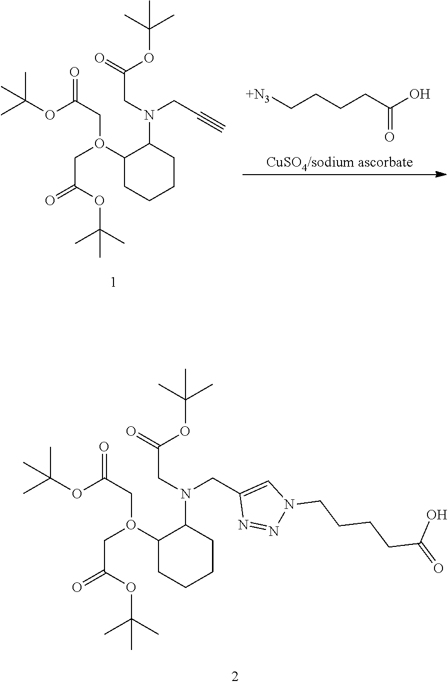

¹H NMR (400 MHz, CDCl₃) δ 8.01, 5.20, 4.34, 3.95, 3.79, 3.70, 3.38, 3.30, 2.61, 2.38, 1.99, 1.97, 1.69, 1.45, 1.43, 1.41, 1.11. ¹³C NMR (101 MHz, CDCl₃) δ 171.85, 171.69, 124.20, 80.77, 77.20, 67.06, 63.00, 52.97, 52.88, 49.82, 45.39, 29.55, 28.11, 28.06, 25.77, 25.53.

Chemical Formula: C₃₂H₅₅N₅O₈, Molecular Weight: 637.81 g/mol, ESI⁺ m/z: 638 [M+H]⁺.

### Preparation of [2-[bis(carboxymethyl)amino]cyclohexyl]-[[1-(4-carboxybutyl)triazol-4-yl]methyl]-(carboxymethyl)ammonium trifluoroacetate (3)

10 mg (15 μmol) of the tert-butyl ester 2 was dissolved in a mixture of TFA (450 μl) and water (50 μl) and stirred for 5 h at rt. Then the reaction mixture was added dropwise to ice cooled Et₂O (10 ml). The supernatant was decanted and the precipitate was washed twice with cold Et₂O. Yield: 5.4 mg (9 μmol; 60%),

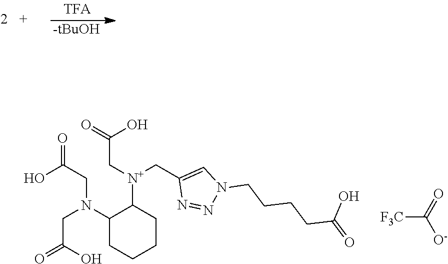 [SMILES: "-tBuOH" ; O=C(O)CCCCn1cc(C[NH+](CC(=O)O)C2CCCCC2N(CC(=O)O)CC(=O)O)nn1 ; O=C([O-])C(F)(F)F]

¹H NMR (400 MHz, D₂O) δ 8.12, 4.47, 4.36, 3.81, 3.44, 3.42, 3.30, 2.29, 2.14, 1.96, 1.83, 1.76, 1.72, 1.43, 1.19, 1.05. ¹³C NMR (101 MHz, D₂O) δ 178.09, 162.67, 127.71, 117.97, 114.76, 65.93, 64.09, 50.27, 32.80, 28.49, 24.16, 23.62, 20.95, 13.99.

Chemical Formula: [C₂₀H₃₂N₅O₈]⁺[C₂F₃O₂]⁻, Molecular Weight(cation): 470.50 g/mol, ESI⁺ m/z: 471 [M+H]⁺.

Preparation of Other Chelators (4-6) of General Formula (III):

Other chelator compounds were synthesized in the same way by copper click reaction of the alkyne 1 with different azido derivatives and subsequent acid deprotection with TFA.

### N-((1-benzyl-1H-1,2,3-triazol-4-yl)methyl)-2-(bis(carboxymethyl)amino)-N-(carboxymethyl)cyclohexan-ammonium trifluoroacetate (4)

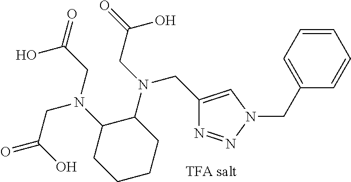 [SMILES: O=C(O)CN(CC(=O)O)C1CCCCC1N(CC(=O)O)Cc1cn(Cc2ccccc2)nn1]

Starting material: Alkyne 1 and (azidomethyl)benzene. ¹H NMR (400 MHz, CD₃CN) δ 8.06, 7.42, 7.40, 7.38, 7.36, 7.32, 7.30, 5.59, 4.56, 4.53, 4.47, 4.13, 4.06, 3.91, 3.87, 3.58, 3.34, 2.87, 2.06, 1.95, 1.95, 1.94, 1.78, 1.56, 1.42, 1.28. ¹³C NMR (101 MHz, CD₃CN) δ 171.67, 169.51, 167.23, 159.29, 159.08, 158.69, 158.13, 135.27, 128.96, 128.50, 127.98, 127.24, 119.35, 117.29, 114.08, 112.10, 65.24, 60.15, 54.30, 53.80, 49.34, 48.36, 24.73, 24.10, 23.79, 23.71. Chemical Formula (cation): C₂₂H₃₀N₅O₆⁺ Molecular Weight: 460.51 g/mol, ESI⁺ m/z: 461 [M+H]⁺

### 2-(bis(carboxymethyl)amino)-N-((1-(2-((5-carboxy-5-(3-(1,3-dicarboxypropyl)ureido)pentyl)amino)-2-oxoethyl)-1H-1,2,3-triazol-4-yl)methyl)-N-(carboxymethyl)cyclohexan-ammonium trifluoroacetate (5)

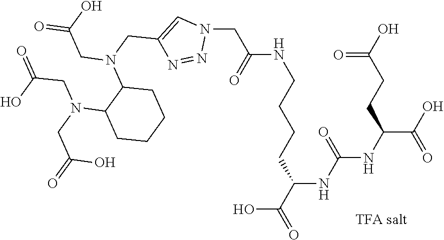 [SMILES: O=C(O)CC[C@H](NC(=O)N[C@@H](CCCCNC(=O)Cn1cc(CN(CC(=O)O)C2CCCCC2N(CC(=O)O)CC(=O)O)nn1)C(=O)O)C(=O)O]

Starting material: Alkyne 1 and di-tert-butyl ((6-(2-azidoacetamido)-1-(tert-butoxy)-1-oxohexan-2-yl)carbamoyl)glutamate. ¹H NMR (400 MHz, D₂O) δ 8.10, 4.65, 4.37, 4.34, 4.18, 4.17, 4.16, 4.14, 4.10, 4.09, 4.08, 4.07, 3.60, 3.56, 3.17, 3.15, 3.14, 2.43, 2.41, 2.39, 2.12, 2.09, 2.06, 2.05, 1.90, 1.88, 1.75, 1.61, 1.45, 1.44, 1.30, 1.20, 1.07. ¹³C NMR (101 MHz, D₂O) δ 177.23, 177.12, 176.27, 167.32, 159.30, 62.55, 53.10, 52.56, 52.27, 39.24, 30.44, 30.03, 27.51, 26.17, 24.18, 23.74, 22.12. Chemical Formula(cation): C₂₉H₄₅N₈O₁₄⁺ Molecular Weight: 728.72 g/mol, ESI⁺ m/z: 729 [M+H]⁺

### N-((1-TATE-1H-1,2,3-triazol-4-yl)methyl)-2-(bis(carboxymethyl)amino)-N-(carboxymethyl)cyclohexan-ammonium trifluoroacetate (6)

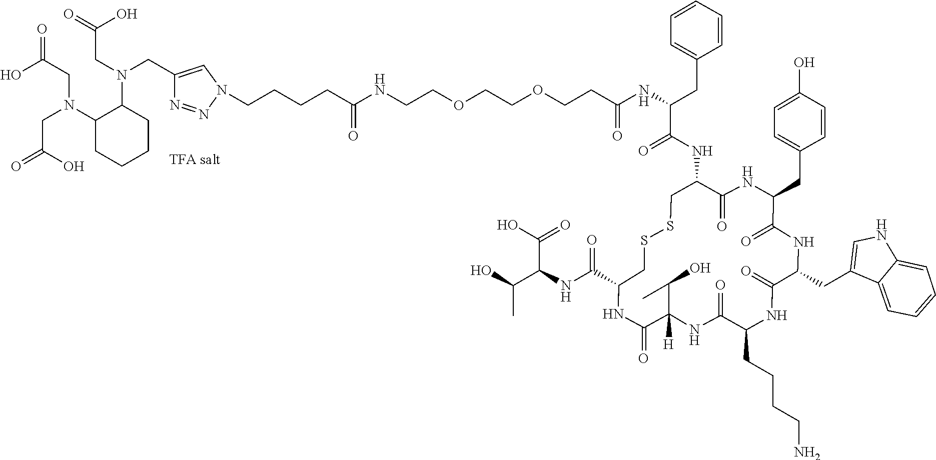 [SMILES: C[C@@H](O)[C@H](NC(=O)[C@@H]1CSSC[C@H](NC(=O)[C@@H](Cc2ccccc2)NC(=O)CCOCCOCCNC(=O)CCCCn2cc(CN(CC(=O)O)C3CCCCC3N(CC(=O)O)CC(=O)O)nn2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Starting material: Alkyne 1 and azidopentanamido [Tyr3]octreotate (TATE-derivative: (2S,3R)-2-[[(4R7S,10S,13R,16S,19R)-10-(4-aminobutyl)-19-[[(2R)-2-amino-3-phenylpropanoyl]amino]-7-[(1R)-1-hydroxyethyl]-16-[(4-hydroxyphenyl)methyl]-13-(1H-indol-3-ylmethyl)-(3,9,12,15,18-pentaoxo-1,2-dithia-5,8,11,14,17-pentazacycloicosane-4-carbonyl]amino]-3-hydroxybutanoic acid).

Chemical Formula(cation): C₇₇H₁₀₉N₂₂O₁₅S₂⁺ Molecular Weight: 1658.91 g/mol, MALDI-TOF MS⁺ m/z: 1659 [M+H]⁺

General Method for Preparation of the Radiolabeled Complexes of Al¹⁸F, ⁶⁸Ga and ⁶⁴Cu:

Radiolabeling of chelator, e.g. [2-[bis(carboxymethyl)amino]cyclohexyl]-[[1-(4-carboxybutyl)triazol-4-yl]methyl]-(carboxymethyl)ammonium trifluoroacetate, with F-18 as Al-Fluorid, Ga-68 and Cu-64 is performed as follows:

To 50-100 μg of above-named chelator dissolved in 200 μl 0.1 M MES buffer pH 5.5 the prepared solution of the radionuclide was added and mixed for 30 min at room temperature.

Before, Al¹⁸F is freshly prepared by mixing from F-18 sodium fluoride solution in saline and 20 μl of a 0.002 M AlCl₃ solution.

Ga-68 gallium(III) chloride diluted in 0.5 M HCl was used directly after elution from the Ge⁶⁸/Ga⁶⁸ generator.

Cu-64 copper(II) chloride was used as solution in 0.01 M HCl after the copper separation.

The completion of the reaction was confirmed by TLC control (2 M NH₄OAc/Methanol 1:1 (v/v) RP18 material). For all of the tested radionuclides (AlF-18, Ga-68, Cu-64) unreacted radiometal species remain on the start of the TLC stripes (Rf=0), whereas the complexes move with Rf=0.7-1 under these conditions.

M=mol/l.

Radio-TLC examples using 2 M NH₄OAc/Methanol 1:1 (v/v) as eluent and RP18 material:

Complex of F-18 Aluminum fluoride (Rf=0) with above named chelator (3):

(Rf=0.7, Yield 97%):

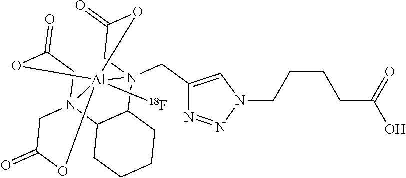

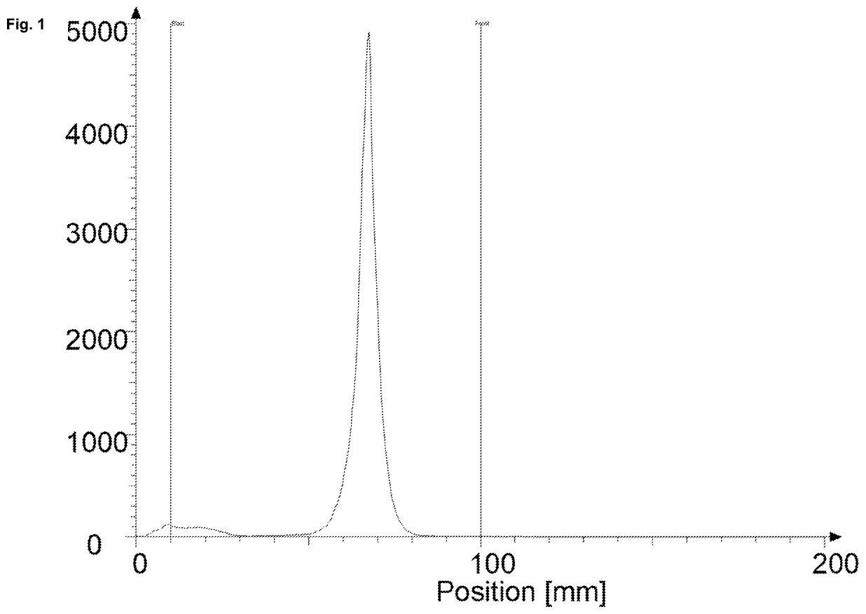

FIG. 1 shows Radio-TLC of the Al¹⁸F-complex.

Complex of Ga-68 with Above Named Chelator (3):

(Rf=1, Yield: 80%):

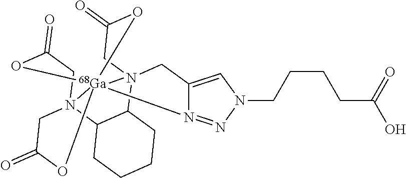

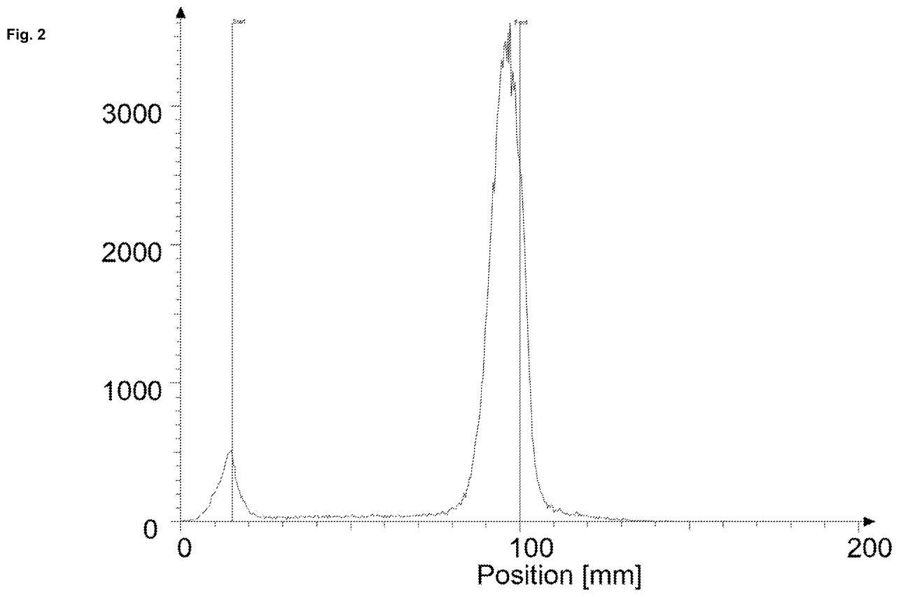

FIG. 2 shows the Radio-TLC of the ⁶⁸Ga-complex.

Complex of Cu-64 with Above Named Chelator (3):

(Rf=0.9, Yield: 99%):

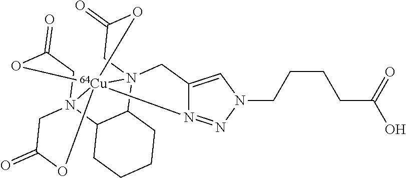

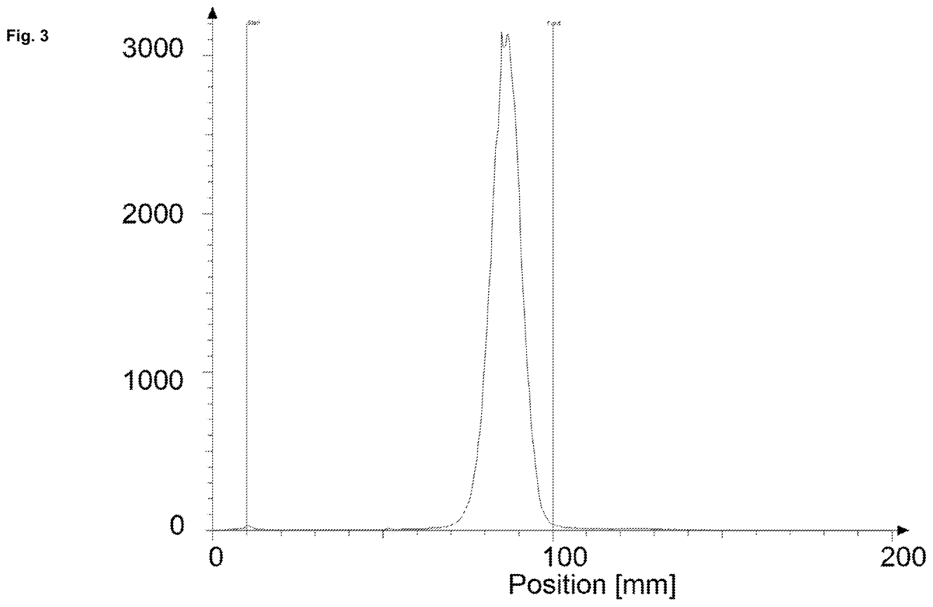

FIG. 3 shows the Radio-TLC of the ⁶⁴Cu-complex.

Additional ligands 4-6 were tested exemplarily only for F-18 labeling as Al—Fluorid complexes:

Complex Comprising Metal Al and Further Ligand ¹⁸F and Chelator (4):

(Rf=0.7, Yield: 99%):

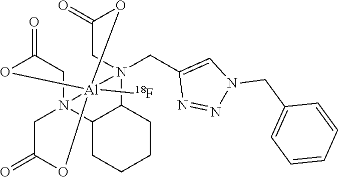

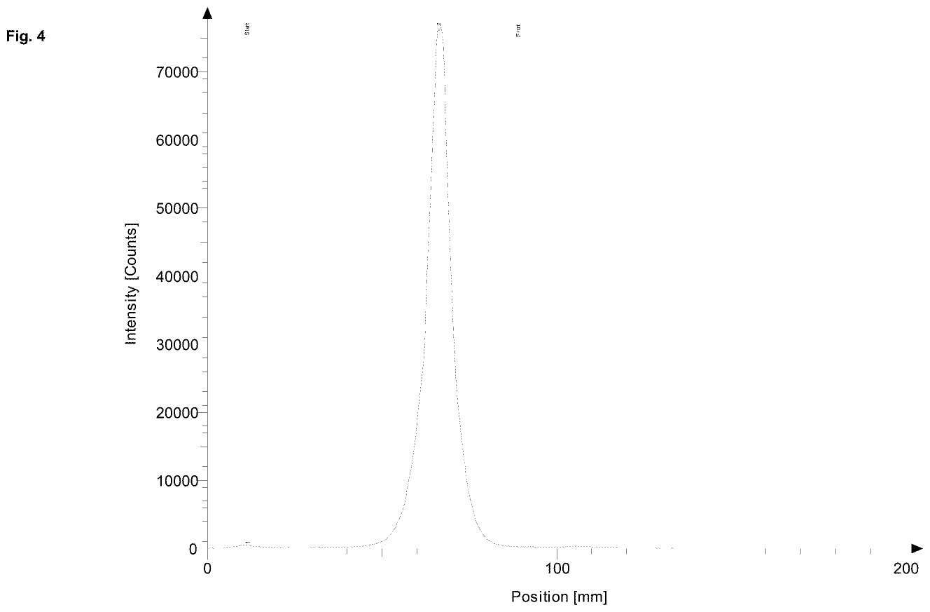

FIG. 4 shows the Radio-TLC of the complex of chelator (4), wherein the metal is Al and the complex further comprises ¹⁸F as a ligand.

Complex Comprising Metal Al and Further Ligand ¹⁸F and Chelator (5):

(Rf=0.9, Yield: 99%):

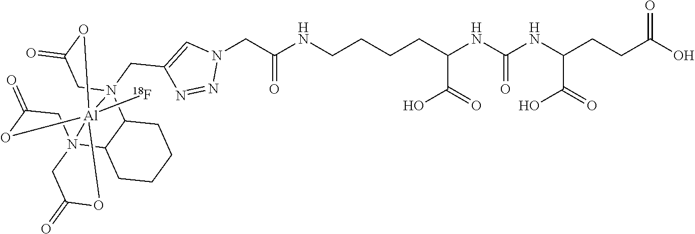

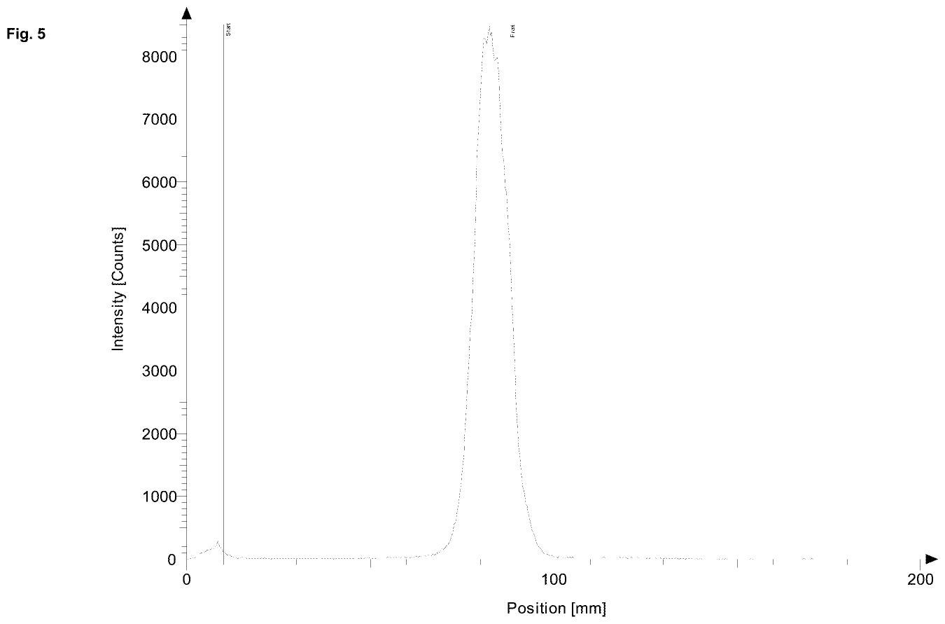

FIG. 5 shows the Radio-TLC of the complex of chelator (5), wherein the metal is Al and the complex further comprises ¹⁸F as a ligand.

Complex Comprising Metal Al and Further Ligand ¹⁸F and Chelator (6):

(Rf=0.8, Yield: 98%):

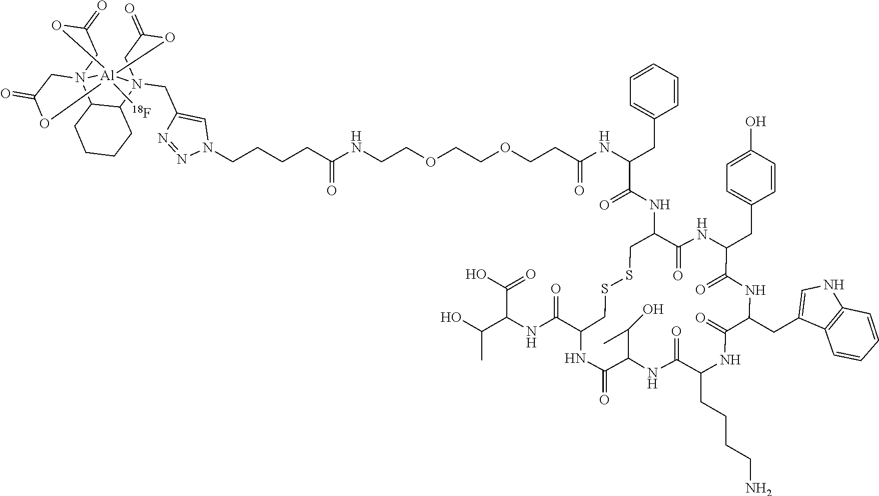

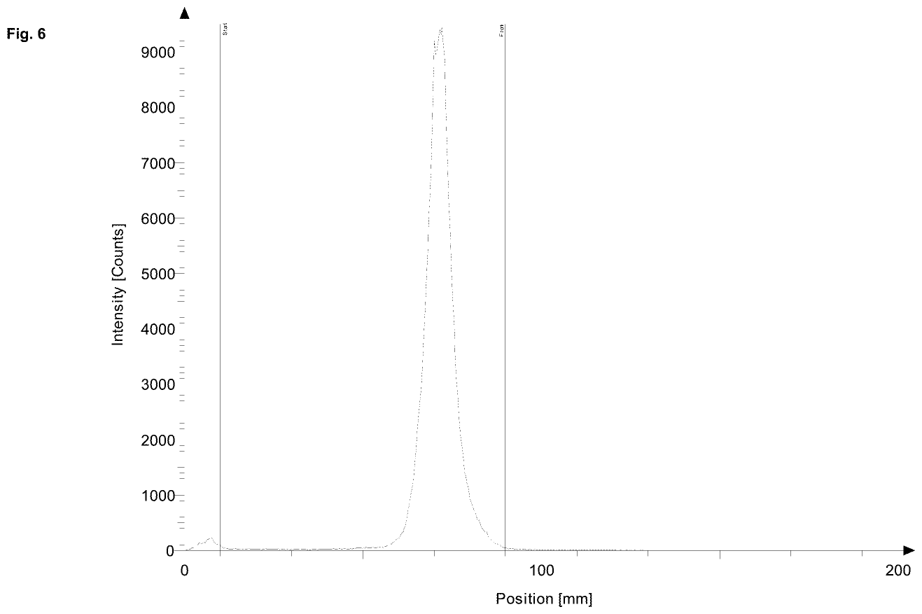

FIG. 6 shows the Radio-TLC of the complex of chelator (6), wherein the metal is Al and the complex further comprises ¹⁸F as a ligand.

Preparation of a Non-Radioactive Kit

The formulation of a non-radioactive kit is produced by mixing all ingredients in an aqueous solution. The formulation may then be sterile filtered, e.g. through a sterile 0.2 μm filter. The formulation is preferably filled into sterile containers. The containers are subsequently sealed and optionally lyophilized. This is preferably performed by first partially sealing the containers, followed by lyophilization and subsequent sealing and capping.

Preparation of a Complex from a Non-Radioactive Kit:

The complex is preferably formed by first adding to the first container, containing chelator of formula (III) or a salt thereof, and optionally a filler, the content of the second container which contains an agent for pH adjustment. In case of a powdery buffer or agent for pH adjustment, a diluent comprising water, preferably water for injections or a saline solution (sterile solution of sodium chloride) is added to the second vial prior to adding the content to the first vial. Subsequently, as an example for Tc-complexes, the pertechnetate solution is added to the mixture of the first and the second vial, resulting in the formation of a pharmaceutical formulation for intravenous administration.

Alternatively, the technetium complex is formed by adding the pertechnetate solution to the first container and immediately of after labeling is finished transferring the content of the second container to the first container.

Biodistribution in SKH1 Mice

The compounds were prepared and complexed with Al¹⁸F as described above.

For the biodistribution two groups of SKH1 mice (n=4) were sacrificed under desflurane anaesthesia at 60 and 240 min, respectively, after injection of 0.30 MBq each. Organs and tissues of interest were removed, weighed, and the activity was measured in a cross-calibrated well counter and dose calibrator. The decay corrected data were normalized to the amount of injected activity calculated from the activity of injection syringes before and after injection and expressed as percentage of injected activity per gram tissue (% ID/g tissue). Values are quoted as mean±standard deviation (mean±+/−SD) for one group of animals.

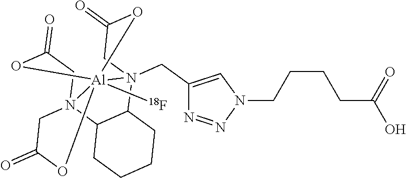

| | 60 min | SD | 240 min | SD |
|---|---|---|---|---|
| % ID/g | | | | |
| blood | 1.19 | 1.01 | 0.23 | 0.42 |
| brown adipose tissue | 0.05 | 0.01 | 0.02 | 0.01 |
| skin | 0.12 | 0.04 | 0.04 | 0.03 |
| brain | 0.01 | 0.00 | 0.00 | 0.00 |
| ovaries | 0.15 | 0.08 | 0.01 | 0.01 |
| uterus | 0.13 | 0.03 | 0.02 | 0.01 |
| pancreas | 0.12 | 0.05 | 0.01 | 0.01 |
| spleen | 0.14 | 0.04 | 0.01 | 0.01 |
| adrenals | 0.22 | 0.11 | 0.03 | 0.03 |
| sum kidneys | 0.37 | 0.08 | 0.03 | 0.01 |
| fat | 0.21 | 0.29 | 0.01 | 0.01 |
| muscle | 0.06 | 0.01 | 0.02 | 0.02 |
| heart | 0.09 | 0.01 | 0.02 | 0.01 |
| sum lung | 0.23 | 0.09 | 0.03 | 0.01 |
| thyroid | 0.25 | 0.09 | 0.38 | 0.49 |
| gall bladder | | | | |
| sum liver | 0.25 | 0.13 | 0.12 | 0.07 |
| femur | 0.36 | 0.05 | 0.37 | 0.09 |
| % ID | | | | |
| intestine | 13.51 | 1.09 | 20.90 | 9.94 |
| urine | 79.26 | 1.31 | 73.24 | 11.75 |

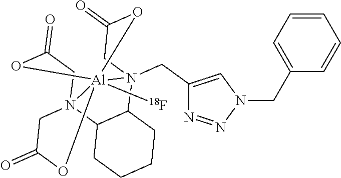

| | 60 min | SD | 240 min | SD |
|---|---|---|---|---|
| % ID/g | | | | |
| blood | 1.99 | 3.91 | 0.02 | 0.01 |
| brown adipose tissue | | 0.13 | 0.03 | 0.02 |
| skin | 0.07 | 0.06 | 0.09 | 0.09 |
| brain | 0.01 | 0.00 | 0.01 | 0.00 |
| ovaries | 0.18 | 0.17 | 0.06 | 0.07 |
| uterus | 0.26 | 0.30 | 0.04 | 0.02 |
| pancreas | 0.20 | 0.33 | 0.04 | 0.02 |
| spleen | 0.14 | 0.25 | 0.02 | 0.02 |
| adrenals | 0.25 | 0.45 | 0.14 | 0.06 |
| sum kidneys | | 0.14 | 0.04 | 0.02 |
| fat | 0.16 | 0.28 | 0.10 | 0.09 |
| muscle | 0.07 | 0.05 | 0.02 | 0.01 |
| heart | 0.07 | 0.10 | 0.01 | 0.01 |
| sum lung | | 0.30 | 0.02 | 0.01 |
| thyroid | 0.35 | 0.40 | 0.18 | 0.10 |
| gall bladder | | | | |
| sum liver | | 1.84 | 0.22 | 0.21 |
| femur | 0.40 | 0.12 | 0.46 | 0.18 |
| % ID | | | | |
| intestine | 51.28 | 1.65 | 54.06 | 7.77 |
| urine | 39.20 | 2.12 | 32.68 | 1.81 |

### LITERATURE

Gale, E. M.; Atanasova, I. P.; Blasi, F.; Ay, I.; Caravan, P.

### A Manganese Alternative to Gadolinium for MRI Contrast

J. Am. Chem. Soc. 137 (2015), 15548-15557
Cleeren, F.; Lecina, J.; Billaud, E. M. F.; Ahamed, M.; Verbruggen, A.; Bormans, G. M.

### New Chelators for Low Temperature Al¹⁸F-Labeling of Biomolecules

### Bioconjugate Chem. 27 (2016), 790-798

Mohamadi, A.; Miller, L. W.
   Efficient route to pre-organized and linear polyaminopolycarboxylates: Cy-TTHA, Cy-DTPA and mono/di-reactive, tert-butyl protected TTHA/Cy-TTHA

### Tetrahedron Letters 58 (2017), 1441-1444

Vanasschen, C.; Molnár, E.; Tircsó, G.; Kálmán, F. K.; Tóth, É.; Brandt, M.; Coenen, H. H.; Neumaier, B.

### Novel CDTA-based, Bifunctional Chelators for Stable and Inert Mn^{II} Complexation:

### Synthesis and Physicochemical Characterization

Inorg. Chem. 56 (2017), 7746-7760

1. A complex comprising a metal M, and a chelator, wherein the chelator is a compound of formula (III) 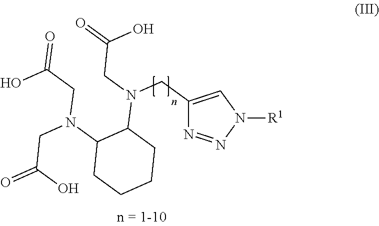
or salts thereof,
wherein R¹ is -alky, -aryl or -heteroaryl, or wherein R¹ is a linker attached either to an active moiety or a group for functionalization.

2. A complex according to claim 1, wherein M is selected from Al, ¹¹¹In, ⁶⁷Ga, ⁶⁸Ga, ⁶⁰Cu, ⁶¹Cu, ⁶²Cu, ⁶⁴Cu, ⁶⁷Cu, ^{99m}Tc, ¹⁸⁶Re, and ¹⁸⁸Re.

3. A complex according to claim 1 further comprising CO, R′NC, ¹⁸F and ═O as ligands, wherein R′ is alkyl or aryl.

4. (canceled)

5. A compound of formula (II) or formula (III), or salts thereof 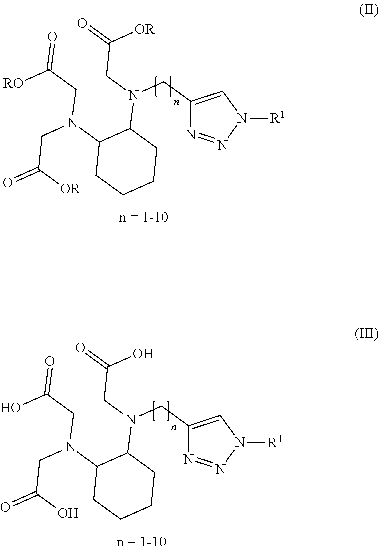
wherein each R is a carboxyl protecting group,
wherein R¹ is -alky, -aryl or -heteroaryl, or a linker attached either to an active moiety or a group for functionalization.

6. Use of one or more of compounds of claim 5 for the preparation of a complex comprising a metal M, and a chelator, wherein the chelator is a compound of formula (III) 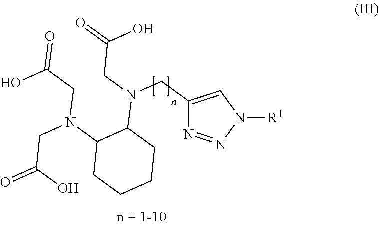
or salts thereof,
wherein R¹ is -alky, -aryl or -heteroaryl, or wherein R¹ is a linker attached either to an active moiety or a group for functionalization.

7. A compound of formula (I) or salts thereof: 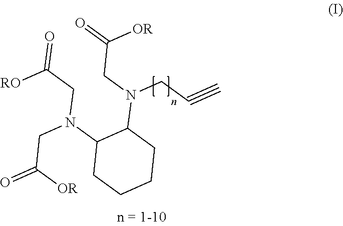
wherein each R is a carboxyl protecting group.

8. Use of a compound according to claim 7 in radiopharmacy.

9. Use of a compound according to claim 7 for preparation of:
a compound of formula (II) or formula (III), or salts thereof 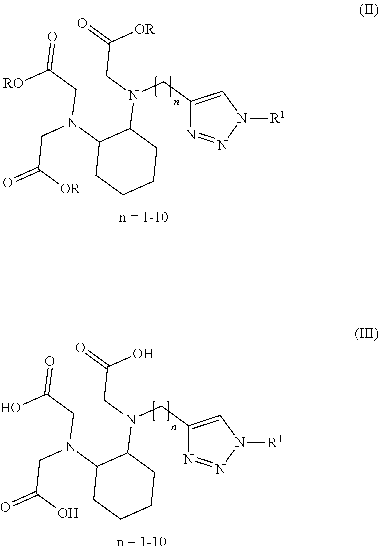
wherein each R is a carboxyl protecting group,
wherein R¹ is -alky, -aryl or -heteroaryl, or a linker attached either to an active moiety or a group for functionalization, or
a complex comprising a metal M, and a chelator, wherein the chelator is a compound of formula (III).

10. A non-radioactive kit for use in radiopharmacy comprising at least one container, wherein the container contains:
(i) a compound of formula (III) according to claim 5.

11. A non-radioactive kit according to claim 10, wherein the kit additionally comprises one or more of the following:
(ii) preservative,
(iii) agent for pH adjustment and
(iv) filler.

12. A complex according to claim 2 further comprising CO, R′NC, ¹⁸F and ═O as ligands, wherein R′ is alkyl or aryl.

13. Use according to claim 6 wherein M is selected from Al, ¹¹¹In, ⁶⁷Ga, ⁶⁸Ga, ⁶⁰Cu, ⁶¹Cu, ⁶²Cu, ⁶⁴Cu, ⁶⁷Cu, ^{99m}Tc, ¹⁸⁶Re, and ¹⁸⁸Re.

14. Use according to claim 13 wherein the complex further comprises CO, R′NC, ¹⁸F and ═O as ligands, wherein R′ is alkyl or aryl.

15. Use according to claim 6 wherein the complex further comprises CO, R′NC, ¹⁸F and ═O as ligands, wherein R′ is alkyl or aryl.

16. Use according to claim 9 for preparation of a complex comprising a metal M, and a chelator, wherein the chelator is a compound of formula (III), wherein M is selected from Al, ¹¹¹In, ⁶⁷Ga, ⁶⁸Ga, ⁶⁰Cu, ⁶¹Cu, ⁶²Cu, ⁶⁴Cu, ⁶⁷Cu, ^{99m}Tc, ¹⁸⁶Re, and ¹⁸⁸Re.

17. Use according to claim 16 wherein the complex further comprises CO, R′NC, ¹⁸F and ═O as ligands, wherein R′ is alkyl or aryl.

18. Use according to claim 9 wherein the complex further comprises CO, R′NC, ¹⁸F and ═O as ligands, wherein R′ is alkyl or aryl.
